(19) United States
(12) Patent Application Publication
(10) Pub. No.: US 2024/0047084 A1
(43) Pub. Date: Feb. 8, 2024

(54) MEDICAL DEVICE SYSTEM FOR CLASSIFICATION AND PREDICTION OF MEDICAL EVENTS USING TIME-BETWEEN-EVENT VALUES

(71) Applicant: Medtronic, Inc., Minneapolis, MN (US)

(72) Inventor: John Dubuc, Scottsdale, AZ (US)

(21) Appl. No.: 18/362,382

(22) Filed: Jul. 31, 2023

Related U.S. Application Data
(60) Provisional application No. 63/370,596, filed on Aug. 5, 2022.

(51) Int. Cl. G16H 80/00; G16H 40/67; G16H 50/30

(52) U.S. Cl. CPC G16H 80/00; G16H 40/67; G16H 50/30

(57) ABSTRACT

A computing system including a memory, processing circuitry coupled to the memory, and communications circuitry. The processing circuitry is configured to determine a time-between-event (TBE) value for a first plurality of events detected by a computing device, determine a classification condition based on the determined TBE value and reference data stored in the memory, the reference data comprising information corresponding to prior detected events, based on a determined change in the TBE value, apply the classification condition to apply a to a plurality of detected events, wherein the plurality of detected events comprises the first plurality of events and the second plurality of events, and determine whether one or more of the plurality of detected events satisfies the classification condition. The communications circuitry is configured to communicate to another device via a network that the one or more of the plurality of detected events satisfies the classification condition.

This application claims the benefit of U.S. Provisional Patent Application No. 63/370,596, filed Aug. 5, 2022 and entitled “MEDICAL DEVICE SYSTEM FOR CLASSIFICATION AND PREDICTION OF MEDICAL EVENTS USING TIME-BETWEEN-EVENT VALUES,” the entire contents of which is incorporated herein by reference.

### TECHNICAL FIELD

This disclosure relates to medical device systems and, more particularly, to medical device systems for detection of events.

### BACKGROUND

In some situations, medical professionals may use a medical device system to track occurrences of events (e.g., medical events). The medical device system may generate control charts to determine changes in the occurrences of the events over time.

### SUMMARY

The devices, systems, and techniques of this disclosure generally relate to the monitoring of trends in detected events. In some examples, a computing system may determine changes in time-between-event (TBE) values between detected events and apply a proportion-based (based on counts or rates) and/or a time-based rareness classification condition. The computing system may determine if changes in the TBE value satisfies a classification condition (e.g., TBE value is outside a control limit or has detected a trend). The computing system may notify a user (e.g., a medical professional) the classification condition has been satisfied. In some examples, the computing system may predict occurrences of future events based on the time-based rareness classification condition.

In an example, the disclosure describes a computing system comprising: a memory; processing circuitry coupled to the memory, the processing circuitry configured to: determine a time-between-event (TBE) value for a first plurality of events detected by a computing device; determine a classification condition based on the determined TBE value and reference data stored in the memory, the reference data comprising information corresponding to prior detected events; determine a change in the TBE value over time for a second plurality of events detected by the computing device; based on the determined change in the TBE value, apply the classification condition to a plurality of detected events, wherein the plurality of detected events comprises the first plurality of events and the second plurality of events; and; and determine whether one or more of the plurality of detected events satisfies the classification condition; and communications circuitry configured to: communicate to another device via a network that the one or more of the plurality of detected events satisfies the classification condition.

In another example, the disclosure describes a computing system comprising: a memory; processing circuitry coupled to the memory, the processing circuitry configured to: determine a time-between-event (TBE) value for a first plurality of symptom occurrences experienced by a patient that are detected by a medical device; determine a classification condition based on the determined TBE value and reference data stored in the memory, the reference data comprising information corresponding to prior detected symptom occurrences; determine a change in the TBE value over time for a second plurality of symptom occurrences experienced by the patient that are detected by the medical device; based on the determination of the change in the TBE value, apply the classification condition to a plurality of detected symptom occurrences, wherein the plurality of detected symptom occurrences comprises the first plurality of detected symptom occurrences experienced by the patient and the second plurality of detected symptom occurrences experienced by the patient; and; and determine whether one or more of the plurality of detected symptom occurrences satisfies the classification condition; and communications circuitry configured to: communicate to another device via a network that the one or more of the plurality of detected symptom occurrences satisfies the classification condition.

In another example, the disclosure describes a method comprising: determining, by processing circuitry of a computing system, a time-between-event (TBE) value for a first plurality of events detected by a computing device; determining, by the processing circuitry, a classification condition based on the determined TBE value and reference data stored in memory of the computing system, the reference data comprising information corresponding to prior detected events; determining, by the processing circuitry, a change in the TBE value over time for a second plurality of events detected by the computing device; applying, by the processing circuitry and based on the determined change in the TBE value, the classification condition to a plurality of detected events, wherein the plurality of detected events comprises the first plurality of events and the second plurality of events; determining, by the processing circuitry, whether one or more of the plurality of detected events satisfies the classification condition; and communicating, by communications circuitry of the computing system, an indication that the one or more of the plurality of detected events satisfy the classification condition to another device via a network.

In another example, the disclosure describes a computer readable storage medium storing instructions that, when executed by processing circuitry within a computing system, cause the processing circuitry to perform a method comprising: determining, by processing circuitry of a computing system, a time-between-event (TBE) value for a first plurality of events detected by a computing device; determining, by the processing circuitry, a classification condition based on the determined TBE value and reference data stored in memory of the computing system, the reference data comprising information corresponding to prior detected events; determining, by the processing circuitry, a change in the TBE value over time for a second plurality of events detected by the computing device; applying, by the processing circuitry and based on the determined change in the TBE value, the classification condition to a plurality of detected events, wherein the plurality of detected events comprises the first plurality of events and the second plurality of events; determining, by the processing circuitry, whether one or more of the plurality of detected events satisfies the classification condition; and communicating, by communications circuitry of the computing system, an indication that the one or more of the plurality of detected events satisfy the classification condition to another device via a network.

In another example, the disclosure describes A computing system comprising: a memory; processing circuitry coupled to the memory, the processing circuitry configured to: determine a time-between-event (TBE) value for a plurality of events detected by a computing device; determine a classification condition based on the determined TBE value and reference data stored in the memory, the reference data comprising information corresponding to prior detected events; apply the classification condition to the plurality of detected events; and determine whether one or more of the plurality of detected events satisfies the classification condition; and communications circuitry configured to: communicate to another device via a network that the one or more of the plurality of detected events satisfies the classification condition.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a conceptual diagram illustrating a system for monitoring events.

FIG. 2 is a conceptual diagram illustrating a time-based control chart generated by the system of FIG. 1.

FIG. 3 is a conceptual diagram illustrating a proportions-based control chart generated by the system of FIG. 1.

FIG. 4 is a conceptual diagram illustrating a computing system of the system of FIG. 1.

FIG. 5 is a conceptual diagram illustrating an example user-complaint system connected to the system of FIG. 1.

FIG. 6 is a flowchart illustrating an example process of monitoring changes in detected TBE events.

FIG. 7 is a flowchart illustrating an example process of determining the presences of changes in TBE values that satisfy a classification condition.

### DETAILED DESCRIPTION

Techniques of this disclosure relate to monitoring of trends in events detected by a computing system. A computing system may detect occurrences of selected events and store data related to the occurrences. The events may include, but are not limited to, application of a medical treatment by a medical device, sensing of symptoms in a patient by a medical device, sensing of a threshold condition by a medical device, occurrence of a medical emergency, occurrence of user complaints, or the like. While the examples of this disclosure are described with respect to the detection of medical events, the examples and techniques of this disclosure may be applied to other event occurrences including, but is not limited to, medical treatment application, monitoring of user complaints, or the like. The computing system may generate a control chart to illustrate changes in the occurrences of the events over time. The control chart may illustrate adverse changes in the occurrences of selected events, e.g., when the events start to increase in frequency.

Control charts, also referred to as “statistical process control charts,” track changes in detected events (also referred herein as “detected events”) over time. Control charts may include proportion-based control charts (e.g., a Laney P′ chart) and time-based control charts (e.g., a T-chart). Proportion-based control charts may be based on counts or rates of occurrences of detected events. Each control chart may include a baseline value (BL), an upper control limit (UCL), and a lower control limit (LCL). In some examples, UCL and LCL may be probability based, (e.g., based on a standard deviation from the baseline value) and/or based one or more recommendations (e.g., based on recommendation(s) from subject-matter experts). The computing system may determine BL, UCL, and LCL based on past recorded data points. Data points are plotted on the control chart, each data point corresponding to a detected event. If the computing system determines that a data point falls outside of an area on the control chart defined by the UCL and the LCL, or a trend in the events, the computing system may determine adverse changes in the detected events and may output a notification to a user indicating the adverse changes.

Proportion-based control charts monitor the proportion of the events that are detected by the computing system relative to a function of the computing system. If the proportion of events for a given time period exceeds the UCL or is below the LCL, or shows a trend, the computing system may detect the presence of adverse changes in the detected events and notify the user. Similarly, time-based control charts illustrate time-between-events (TBE) values as data points. If a TBE value falls outside of the area defined by UCL and LCL, or shows a trend, the computing system may detect the presence of adverse changes in the detected events and notify the user.

For each control chart, the computing system may determine and adjust the BL, UCL, and LCL based on the recorded data points. In some examples, when event occurrences are “rare” (e.g., an average of one or less detected events per month) and the detected events increase in frequency, the computing system may miss adverse changes in the detected events. For example, proportion-based control charts may not detect adverse trends where the proportionality of the detected events forming the adverse trend do not exceed the UCL or are not below the LCL. In some examples, the computing system needs to record data points over a long period of time (e.g., a plurality of months or events) to determine the BL, UCL, and/or LCL needed to setup the control chart and then to adequality detect adverse signals. For example, time-based control charts may use the BL and recorded data points to determine the UCL and/or the LCL and may create an excessively wide range defined by the UCL and the LCL.

The devices, systems, and techniques of this disclosure may provide increased responsiveness (e.g., earlier detection) in determining the adverse changes in the detected events. A computing system in accordance with this disclosure may determine a BL, UCL, and/or LCL of a control chart using two or more data points, which reduces the time required to generate a control chart and/or the responsiveness of the control chart to changes in the frequency of detected events. The computing system may also increase the responsiveness of the determinations of the computing system relative to proportion-based control charts (e.g., Laney P′Chart) or time-based control charts (e.g., T-Chart) alone. A computing system in accordance with this disclosure may determine UCL and/or LCL that are more accurate, responsive, and/or appropriate than those determined by proportion-based control charts or time-based control charts alone and may reduce the likelihood and/or frequency of false alarms. In some examples, a computing system in accordance with this disclosure may provide an indication for an adverse trend in the detected events which may not be detected by proportion-based control charts or time-based control charts alone.

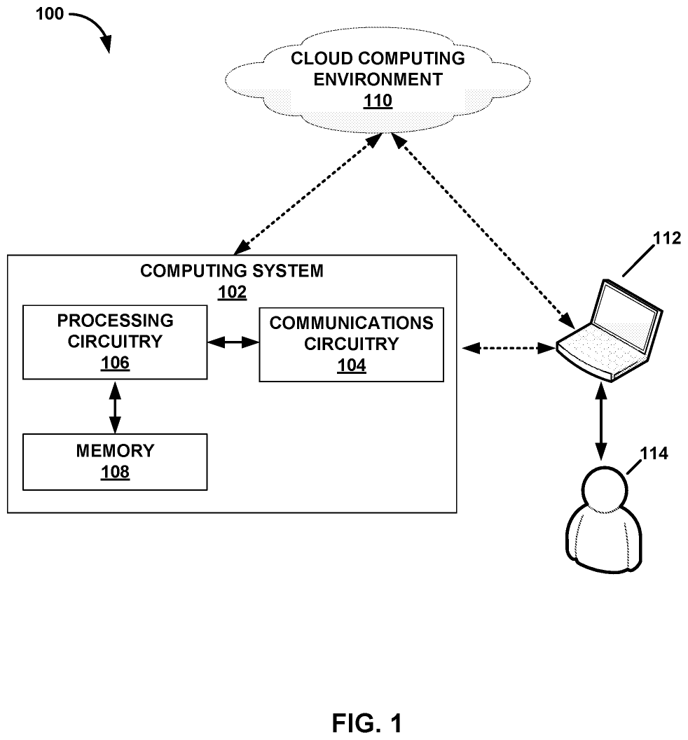

FIG. 1 is a conceptual diagram illustrating a system 100 for monitoring events. System 100 includes a computing system 102, a computing device 112, and a cloud computing environment 110. Computing device 112 may detect and record detected events from user 114. Computing device 112 may include, but is not limited to, cellphones, smartphones, tablets, laptops, personal computers, or smartwatches. Computing device 112 may transmit the record detected events and data corresponding to the detected events to computing system 102 and/or cloud computing environment 110.

In some examples, user 114 is a patient and computing device 112 is a medical device (e.g., an external medical device, an implantable medical device (IMD), or the like) configured to transmit or receive information from to or from user 114. Computing device 112 may be configured to detect and record occurrences of symptoms experienced by user 114. In some examples, computing device 112 may detect and record administration of one or more medical treatments to user 114. For example, when computing device 112 is or is connected to an implantable cardiac device (e.g., a pacemaker or the like), computing device 112 may record delivery of stimulus to heart of user 114. For each detected event, computing device 112 may record data corresponding to the detected event, the data including, but is not limited to, a time of the occurrence, a type of the event (e.g., a symptom, a treatment, or the like), or a severity of the event. While FIG. 1 illustrates a single computing device 112, other examples may include multiple computing devices 112 configured to receive information from multiple users 114 or a single user 114.

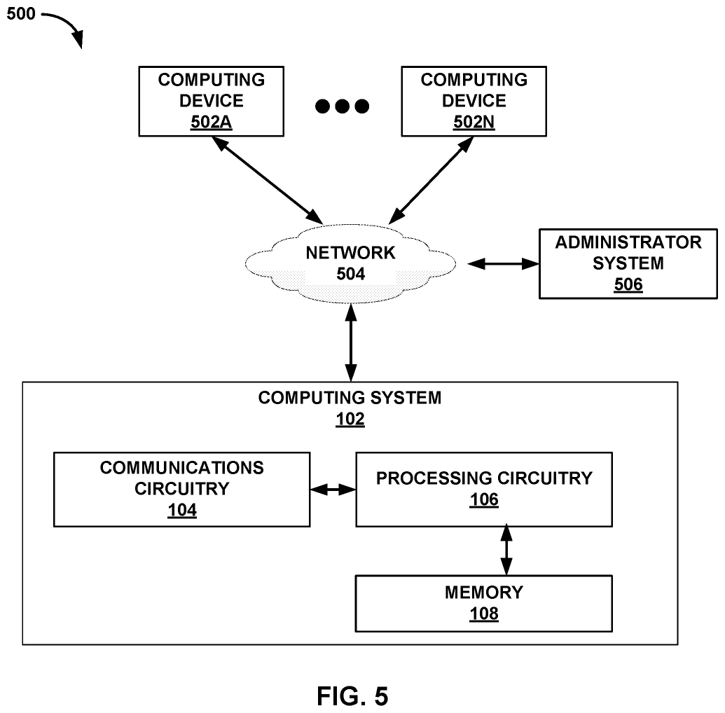

In some examples, computing device 112 may be configured to receive inputs from user 114 corresponding to detected events. Computing device 112 may include a user interface (UI) (not pictured) configured to receive input, e.g., from user 114, or another user. Examples of input include tactile, audio, kinetic, or optical input. User 114 may input to computing device 112, via the UI, information corresponding to detected events (e.g., a time of the occurrence, a type of the occurrence, a severity of the occurrence, or the like). In some examples, when system 100 is incorporated into a user-complaint system (discussed in greater detail in FIG. 5), user 114 may enter a complaint and/or issue into computing device 112 and computing device 112, computing system 102, and/or cloud computing environment 110 may determine the corresponding detected event information based on the user input.

Computing device 112 may, upon receiving the inputs from user 114 corresponding to detected events, record information of the detected events (also referred to as “event information”) including, but are not limited to, whether an event occurred, times of the detected events, types of the detected events, or severity of the detected events. Computing device 112 may transmit the event information to computing system 102 and/or cloud computing environment 110. Computing system 102 and/or cloud computing environment 110 may store the event information.

Computing system 102 may include communications circuitry 104, processing circuitry 106 and memory 108. Computing system 102 may be one or more computing devices and/or one or more computing systems. In some examples, computing system 102 may include additional components. In some examples, computing system 102 may be a part of cloud computing environment 110. In some examples, cloud computing environment 110 may perform the functions of computing system 102 described below.

Communications circuitry 104 may be configured to communicate with computing device 112 and/or cloud computing environment 110. Communications circuitry 104 may receive and/or transmit the event information. Communications circuitry 104 may include a radio transceiver configured for communication according to standards of protocols, such as 3G, 4G, 5G, WiFi (e.g., 802.11 or 802.15 ZigBee), Bluetooth®, or Bluetooth® Low Energy (BLE).

Memory 108 may be configured to store event information received by computing system 102, e.g., via communications circuitry 104. Processing circuitry 106 may receive the event information from communications circuitry 104 and transmit the event information to memory 108 for storage within memory 108. Memory 108 may store computer-readable instructions that, when executed by processing circuitry 106, cause computing system 102 to perform various functions. Memory 108 may be a storage device or other non-transitory medium. Memory 108 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Processing circuitry 106 may include any one or more of a microprocessor, a controller, a digital signal processor (DSP), an application-specific integrated circuit (ASIC), a field-programmable gate array (FPGA), discrete logic circuitry, or any other processing circuitry configured to provide the functions attributed to processing circuitry 106 herein and may be embodied as firmware, hardware, software, or any combination thereof.

Processing circuitry 106 may generate one or more control charts based on the event information received from computing device 112, e.g., via communications circuitry 104. Processing circuitry 106 may execute computer-readable instructions stored in memory 108, computer-readable instructions stored in cloud computing environment 110, and/or one or more applications stored in memory 108 and/or cloud computing environment 110 to generate the one or more control charts.

The one or more control charts may include proportion-based control charts and/or time-based control charts. Each of the one or more control charts may have a time of occurrence as an x-axis. Processing circuitry 106 may plot data points onto the one or more control charts, each data point corresponding to a separate detected event or the number of events in a time period. Processing circuitry 106 may plot the data points by the time of the detected event.

Upon receiving the event information, processing circuitry 106 may determine a number of detected events recorded in the event information. In some examples processing circuitry 106 determines detected events based on input of user 114. In some examples, processing circuitry 106 determines detected events based on a determination that sensed data on user 114 from computing device 112 satisfies a detected event condition. For example, a computing device 112 that is a cardiac monitor may sense heart rate data of user 114 and processing circuitry 106 may determine an occurrence of atrial fibrillation based on a determination that the sensed heart rate data satisfies a threshold AF occurrence condition.

For each of the determined detected events, processing circuitry 106 may determine data corresponding to the detected event from the received event information. The data may include, but are not limited to, a time of occurrence, a TBE value relative to the last recorded detected event, or a rate of event occurrences for a given time period (e.g., a week, a month, or the like. A cycle may be any repeating process that computing system 102 and/or computing device 112 is monitoring including, but are not limited to, a patient monitoring period, a customer monitoring period, or the like.

Processing circuitry 106 may generate time-based control charts with a time of occurrence as the X-axis of the chart and the TBE value as the Y-axis of the chart. Processing circuitry 106 may generate proportion-based control charts with the time of occurrence as the X-axis of the chart and the rate of the occurrence as the Y-axis of the chart, e.g., a number of event occurrences per month. Processing circuitry 106 may execute one or more statistical analysis applications (e.g., R, Microsoft Excel, Minitab developed by Minitab, LLC, or the like) to generate the time-based control charts and/or proportion-based control charts.

Processing circuitry 106 may determine, for each of the control charts, a BL, a UCL, and a LCL. Processing circuitry 106 may plot BL, UCL, and LCL onto the control charts. In some examples, processing circuitry 106 may adjust BL, UCL, and LCL over time as processing circuitry 106 receives more event information and identifies more detected events. For example, if processing circuitry 106 determines a second plurality of detected events with TBE values that are different from the BL, processing circuitry 106 may adjust the BL, UCL, and LCL based at least in part on the TBE values of the second plurality of detected events. UCL and LCL define a range of acceptable data values (e.g., TBE values, event rate values, or the like) for detected events. In some examples, processing circuitry 106 transmits an indication to a user, e.g., via a display device of computing system 102 (not shown), computing device 112, and/or another computing device and/or system connected to cloud computing environment 110. The indication may be visual or auditory and may communicate to user 114 that one or more detected events are detected outside of the control limits (either UCL or LCL) or that a trend is present.

For a time-based control chart, processing circuitry 106 may determine a BL using two or more detected events. Processing circuitry 106 may then determine a UCL and a LCL based on the determined BL. In some examples, computing system 102 may receive reference data corresponding to past monitoring of similar events from cloud computing environment 110. Processing circuitry 106 may determine the UCL and the LCL based on the reference data. The reference data may include, but are not limited to, past detected events and accompanying data stored in cloud computing environment 110. In some examples, processing circuitry 106 may determine that detected events within a percentage of deviation of the TBE BL value (e.g., ±30% of the TBE BL value, a function of the TBE BL value, or the like) are acceptable and may determine the UCL and the LCL based on the percentage. In such examples, the UCL may be +30% of the TBE BL value (i.e., 130% of TBE BL value and the LCL may be −30% of the TBE BL value (i.e., 70% of the TBE BL value). Processing circuitry 106 may generate the control chart with two or more detected events and the reference data.

In some examples, depending on the type of event computing system 102 is monitoring, processing circuitry 106 may set UCL and/or LCL as a classification condition. In some examples, processing circuitry 106 determines that a detected event satisfies the classification condition if the data value of the event is greater than the UCL or is below the LCL. Computing system 102 may output a notification, e.g., in accordance with the process described above, based on a determination that the classification condition is satisfied. In some examples, processing circuitry 106 may identify events that satisfy the classification condition onto the control charts.

Processing circuitry 106 may determine trends in the detected events over time. The trends may include an increase/decrease in frequency of detected events, an increase/decrease in the severity of detected events, or the like. In some examples, processing circuitry 106 may determine changes in trends of the detected events and may adjust BL, UCL, and/or LCL higher and/or lower in response to the determined changes. In some examples, processing circuitry 106 determines or predicts whether the next event is within the control limits (UCL, LCL) or some other range based on the current date/time and the last observed event date/time.

In some examples, once processing circuitry 106 generates the one or more control charts, processing circuitry 106 may send the one or more control charts to memory 108 for storage and/or to communications circuitry 104 for transmittal to computing device 112, cloud computing environment 110, and/or one or more other computing devices and/or computing systems, e.g., connected to cloud computing environment 110.

In some examples, processing circuitry 106 may detect one or more events based on event information stored in memory 108 of computing system 102 without generating the one or more control charts. Processing circuitry may set UCL and/or LCL as threshold conditions and may process event information of detected events to determine if one or more of the detected events satisfies a threshold condition (e.g., is greater than or equal to UCL or less than or equal to LCL). Based on a determination that at least one detected event satisfies a threshold condition, Computing system 102 may output one or more notifications, e.g., via communications circuitry 104, to computing device 112, cloud computing environment 110, and/or one or more other computing devices and/or computing systems, e.g., connected to cloud computing environment 110.

The one or more notifications may include event information of the detected events that satisfy a threshold condition. The event information may include outlier and/or trend detection criteria for detection of one or more trends by computing system 102. In some examples processing circuitry 106 processes the event information of the detected events that satisfy a threshold condition and determines, based on the process event information, occurrences of future detected events. In such examples, the one or more notifications may include a predicted or expected range of time when the next future event will occur.

Cloud computing environment 110 may perform at least some of the functions of computing system 102 and/or processing circuitry 106, e.g., as described above. Cloud computing environment 110 may facilitate connection between computing system 102, computing device 112, and/or one or more other computing devices and/or computing systems. Cloud computing environment 110 may receive, transmit, and/or save event information, control charts, classification conditions, computer-readable instructions, indications that one or more detected events satisfy a classification condition, or the like.

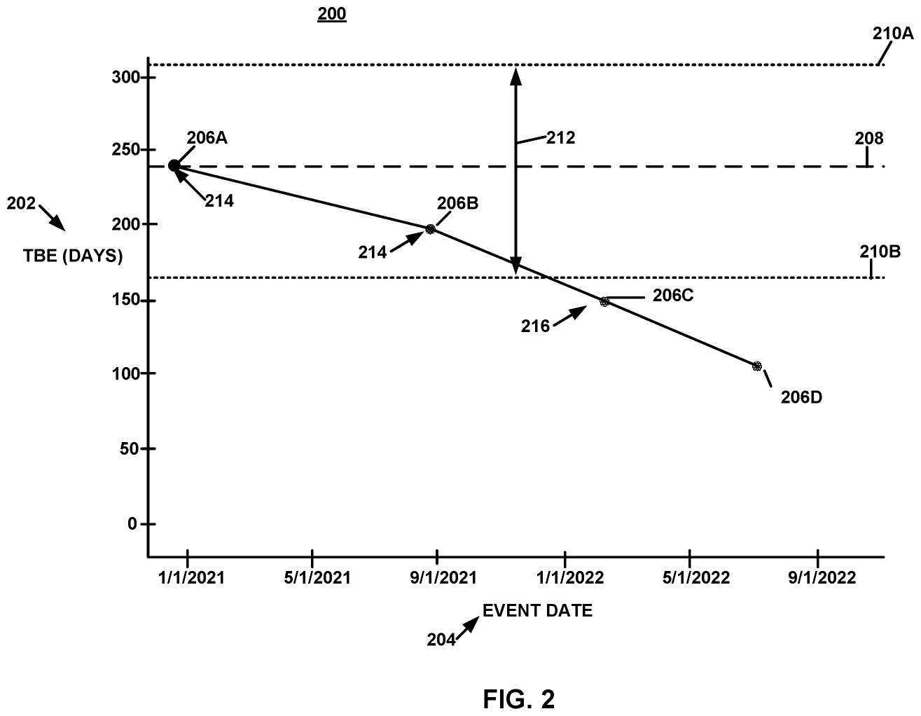

FIG. 2 is a conceptual diagram illustrating a time-based control chart 200 (also referred to as “control chart 200”) generated by the system 100 of FIG. 1. As illustrated in FIG. 2, control chart 200 displays event times 204 (also referred to as “time of detected event”) on the X-axis and the corresponding TBE value 202 on the Y-axis. Event times 204 may be a time of occurrence of the corresponding event. Control chart 200 displays detected events as events 206A-D (collectively referred to as “events 206”). Control chart 200 may define each of events 206 based on the corresponding event times 204 and TBE values 202. The TBE values 202 for events 206 represent a time between each corresponding event of events 206 and an adjacent, prior event of events 206. While the example control chart 200 illustrated in FIG. 2 represents TBE values 202 in days, other control charts 200 may represent TBE value 202 in months, weeks, hours, minutes, seconds, fractions of seconds, or any other unit of elapsed time. Control chart 200 may display a BL 208. BL 208 may represent an acceptable baseline for a monitored event (e.g., occurrence of a symptom, delivery of a medical therapy, user complaint, or other events). As illustrated in FIG. 2, BL 208 represents an acceptable TBE value 202 between adjacent events of events 206.

System 100 may determine first event 206A on control chart 200 based on the first two detected events detected by system 100. System 100 may determine BL 208 based on the two detected events. In some examples, system 100 determines BL 208 by determining an average TBE value of more than two detected events. System 100 may determine UCL 210A and LCL 210B based at least in part on the two detected events, BL 208, and/or reference data. The reference data may include, but are not limited to, prior detected events of user 114, prior detected events of other individuals, or the like. UCL 210A and LCL 210B defines acceptable region 212. Acceptable region 212 corresponds to TBE values 202 that are within an acceptable or stable range for the event. In some examples, system 100 displays on control chart 200, for each of BL 208, UCL 210A, and LCL 210B, a corresponding TBE value.

In some examples, if one or more of events 206 has a corresponding TBE value 202 outside of acceptable region 212 (e.g., below the LCL 210B), system 100 may label the corresponding TBE value 202 (e.g., via indicator 216). Control chart 200 may include a legend (not pictured) configured to illustrate that indicators 214 correspond to non-adverse events and indicators 216 correspond to adverse events. In some examples, if system 100 determines that one or more of events 206 (e.g., event 206B) is outside of acceptable region 212, system 100 may output a notification, e.g., to cloud computing environment 110 and/or computing device 112. The notification may indicate that the one or more events 206 is an adverse event and may indicate an event date 204 and/or TBE value 202.

In some examples, if TBE values 202 for consecutive events 206 (e.g., three or more consecutive events 206) are decreasing, system 100 may determine presence of a trend (e.g., an adverse trend). System 100 may output a notification indicating the presence of the trend and information corresponding to the trend (e.g., trend detection criteria, event dates 204 and/or TBE values 202 for the consecutive events 206, or the like).

In some examples, such as illustrated in FIG. 2, computing system 102 may be configured to detect events 206 that are harmful and/or detrimental. In such examples, computing system 102 may identify low TBE values 202 (e.g., TBE values 202 below LCL 210B) and/or decreasing trends in TBE values 202 as adverse events and output the notification indicating the presence of adverse events. In other examples, computing system 102 may be configured to detect events 206 that are beneficial and may identify high TBE values 202 (e.g., TBE values 202 above UCL 210A) and/or increase trends in TBE values 202 as adverse events.

In some examples, computing system 102 may determine variations in TBE values 202 of events 206 and may determine the presence of adverse events based on the presence, or absence, of adverse events, depending on the characteristics of events 206. For example, when computing system 102 is configured to detect the presence of irregular cardiac symptoms (e.g., arrhythmia) in a patient, computing system 102 may determine the presence of irregular cardiac symptoms based on a high variation in TBE values 202 (e.g., a change in TBE values 202 between consecutive events 206 that satisfy a threshold variation value).

In some examples, where TBE values 202 exhibit a low variation (e.g., where changes in TBE values 202 between consecutive events 206 do not satisfy a threshold variation value), events 206 may represent a periodic signal with uniform or substantially uniform TBE values 202. Such events 206 may be used (e.g., by a clinician or a monitoring system) to monitor user 114. For example, events 206 separated by relatively uniform TBE values 202 may represent a specific frequency of events 206.

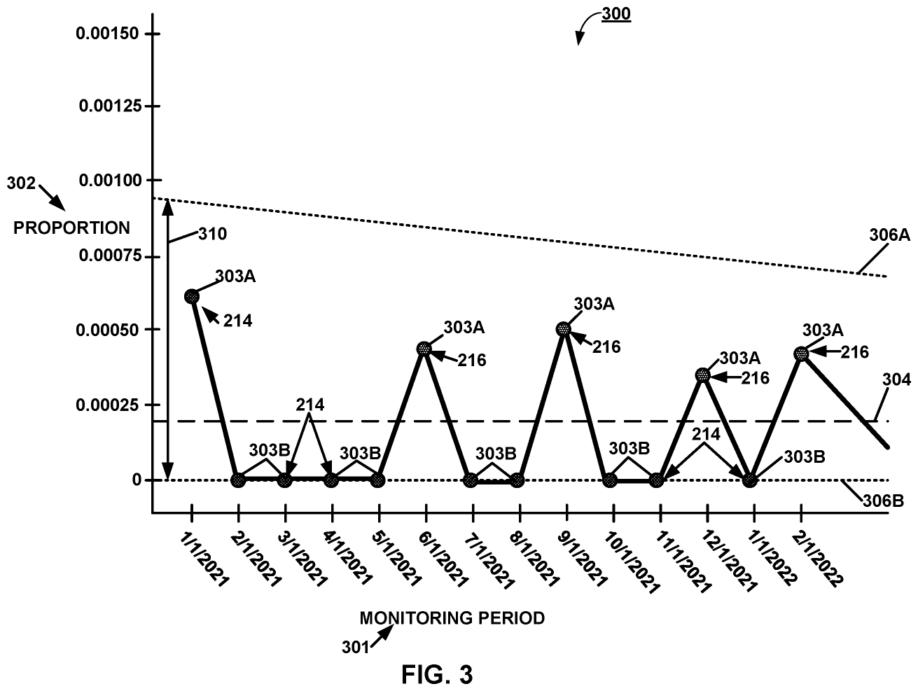

FIG. 3 is a conceptual diagram illustrating a proportion-based control chart 300 generated by the system 100 of FIG. 1. In some examples, control chart 300 and control chart 200 may illustrate the same data set (e.g., a same set of events and corresponding event data). Control chart 300 may display monitoring periods 301 on the X-axis and the proportion 302 (also referred to as “proportionality 302”) on the Y-axis. While this disclosure primarily describes the Y-axis of control chart 300 in terms of proportionality, the Y-axis of control charts generated by system 100 in accordance with the techniques of this disclosure may represent any metric that includes a numerator and a denominator such as, but are not limited to: a percentage of users experiencing an event, a number of events per user, or the like. Proportion 302 may represent a number of events per a population size (e.g., a user population size, a patient population size, a number of devices within a network, or the like) for a given period of time (e.g., within one month as illustrated in FIG. 3. Control chart 300 displays monitoring cycles 303 for each of monitoring period 301. Control chart 300 may define each monitoring cycle 303 based on the monitoring period 301. If system 100 determines one or more event occurrences during a monitoring cycle 303, system 100 may represent the corresponding monitoring period as detected event 303A. If system 100 determines that no event occurred during a monitoring cycle, system 100 may represent the corresponding monitoring cycle 303 as event non-occurrence 303B (e.g., a point at 0 on the Y-axis). The proportionality 302 of each monitoring cycle 303 represents of the number detected events 303A relative to the population size. System 100 may determine BL 304 by recording a plurality of detected events prior to generation of control chart 300. System 100 may determine UCL 306A and LCL 306B based on the recorded detected events. In some examples, system 100 may determine BL 304, UCL 306A, and/or LCL 306B based at least in part on reference data (e.g., prior detected events and/or non-occurrences).

In some examples, control chart 300 may display an acceptable region 310 defined by UCL 306A and LCL 306B. The acceptable region 310 may define a range of acceptable proportionalities 302 of detected events for monitoring cycles 303. If proportionality 302 of a monitoring cycle 303 is outside of acceptable region 310, system 100 may label the corresponding detected event 303A and/or event non-occurrence 303B with indicator 214, e.g., in accordance with the example process described above. If proportionality 302 of a monitoring cycle 303 is outside of acceptable region 310, system 100 may represent the corresponding detected event 303A and/or event non-occurrence 303B with indicator 216, e.g., in accordance with the example process described above. In some examples, control chart 300 includes a legend (not pictured) configured to illustrate that indicator 214 corresponds to non-adverse events and that indicator 216 corresponds to adverse events.

In some examples, if system 100 determines that a detected event 303A and/or event non-occurrence 303B is outside of acceptable region 212, system 100 may output a notification, e.g., to cloud computing environment 110 and/or computing device 112. The notification may indicate that one or more detected events 303A is an adverse event and may indicate an event date 204 and/or a proportionality 302 for each of the one or more detected events 303A.

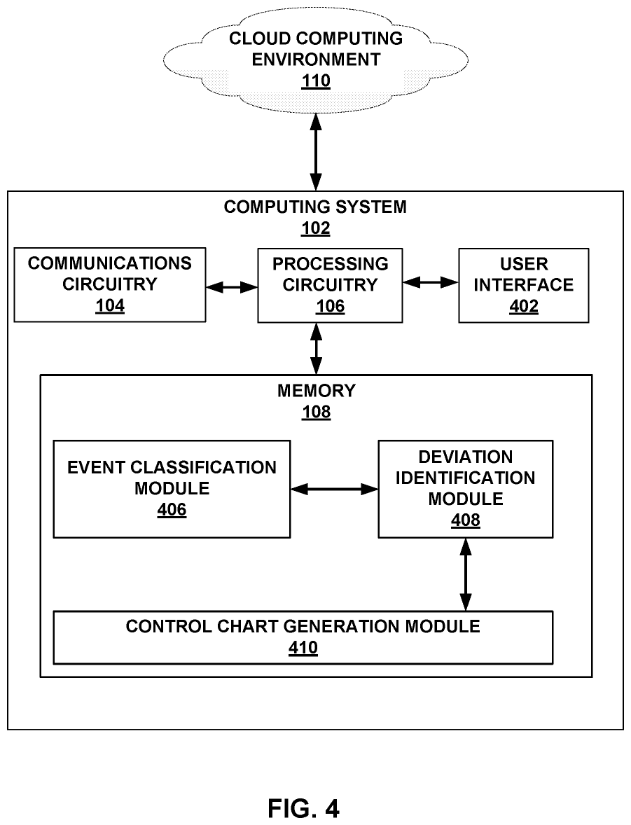

FIG. 4 is a conceptual diagram illustrating a computing system 102 of the system 100 of FIG. 1. As shown in FIG. 4, computing system 102 includes communications circuitry 104, processing circuitry 106, user interface (UI) 402, and memory 108. Processing circuitry 106 and communications circuitry 104 may perform the functions described in FIG. 1.

UI 402 may receive input from one or more users (e.g., user 114). The input may be visual, tactile, and/or auditory input. In some examples, UI 402 may output one or more notifications to the one or more users (e.g., via visual and/or auditory output). The notifications may include one or more indications of occurrences of adverse events, a time (e.g., event date) for each adverse event, a TBE value for each adverse event, a severity of each adverse event, and/or a proportionality of each adverse event. In some examples, UI 402 may receive the input from the one or more users and may transmit the received input to processing circuitry 106. In some examples, UI 402 may transmit the notifications from processing circuitry 106 to the one or more users.

Memory 108 includes computer-readable instructions that, when executed by processing circuitry 106, cause computing system 102 and/or processing circuitry 106 to perform various functions and/or processes. Memory 108 may include event classification module 406, deviation identification module 408, and control chart generation module 410.

Event classification module 406 may store computer-readable instructions that, when executed by processing circuitry 106, cause computing system 102 to detect event occurrences (e.g., e.g., occurrence of a symptom, delivery of a medical therapy, user complaint, or the like). In some examples, Processing circuitry 106 ay determined detected events by determining if recorded data (e.g., from computing device 112) satisfies one or more threshold conditions. In some examples, such as if the monitored event is an occurrence of a symptom, processing circuitry 106 may determine a detected event based on patient data, e.g., from computing device 112 and/or a medical device. Processing circuitry 106 may determine an occurrence of a symptom by determining if patient data satisfies a threshold condition (e.g., presence of an irregular rhythm in electrocardiogram (ECG) of a patient for detection of atrial fibrillation (AF)). In some examples, such as if the monitored event is a delivery of a medical therapy, a user complaint, or the like, processing circuitry 106 may determine a detected event based on information from user 114, computing device 112, and/or cloud computing environment 110 that an event occurred (e.g., a medical device delivered a medical therapy, a user entered a complaint into a computing device 112 and/or cloud computing environment 110, or the like).

Deviation identification module 408 may store computer-readable instructions that, when executed, cause processing circuitry 106 to determine if a detected event (e.g., as detected by processing circuitry 106) exhibits characteristics such that the detected event may be classified as an “adverse event.” An adverse event may be any detected events of a monitored event where one or more characteristics of the detected event fall outside of the allowable ranges of values for the one or more characteristics (e.g., as bounded by UCL (e.g., UCL 210A, UCL 306A) and LCL (e.g., LCL 210B, LCL 306B) of each characteristic). The characteristics may include, but are not limited to, TBE value 202 and proportion 302. Adverse events may also include time-based trends if detected in any range of the Y-axis metric. The time-based trend may be a sequence of decreasing TBE values in the detected events and may be labelled as an “adverse trend.”

Processing circuitry 106 may, after determining that one or more detected events may be classified as an adverse event, transmit a notification to computing device 112, cloud computing environment 110, and/or one or more other computing devices and/or computing system through communications circuitry 104. The notification may indicate event date 204, TBE value 202, and/or proportion 302. In some examples, processing circuitry 106 may transmit one or more recommended changes to one or more activities that may affect the monitored event to reduce the number of future adverse events. The recommended changes may include changes to one or more parameters of a medical therapy or the like. In some examples, in response to the determination of one or more adverse events, computing system 102 may transmit instructions to cloud computing environment 110, computing device 112, and/or one or more other computing devices and/or systems to reduce perform the recommended changes. The recommended changes may include, but are not limited to, changes in the intensity of a medical therapy provided to a patient, changes in the frequency of a medical therapy provided to a patient, recommended medical therapies and/or procedures to a symptom experienced by the patient, recommended workflow changes to reduce the number of detected events, or the like.

In some examples, deviation identification module 408 may store computer-readable instructions that, when executed, cause processing circuitry 106 to predict a future occurrence. Processing circuitry 106 may determine, as a part of predicting a future occurrence, a predicted time and/or an expected time (e.g., an expected range of time) of the detected event. For example, processing circuitry 106 may determine a time of a future event occurrence based at least in part on the TBE value 202 of one or more prior determined detected events. Computing system 102 may transmit the future event occurrences to computing device 112 and/or cloud computing environment 110 (e.g., via communications circuitry 104) and to one or more users (e.g., user 114), e.g., via UI 402. Computer-readable instructions that, when executed, cause processing circuitry 106 to predict future event occurrences and/or transmit the predicted future event occurrences. The future event occurrences may be stored in memory 108, e.g., in deviation identification module 408. In some examples, computing system 102 may determine based on the time of the future occurrence if the detected events are decreasing in frequency. Computing system 102 may then clear one or more alarm states and/or transmit a signal indicating the change (e.g., as an improvement in a medical condition, as a reduction in user complaints, or the like).

In some examples, processing circuitry 106 may predict a future occurrence with a corresponding TBE value greater than the UCL. Processing circuitry 106 may, in response to determining that the future occurrence has a TBE value greater than the UCL, adjust the BL, UCL, and/or LCL based on the prediction (e.g., by increasing the BL, UCL, and/or LCL). In some examples, processing circuitry 106 may instruct communications circuitry 104 to transmit a notification to computing device 112, cloud computing environment 110, and/or one or more other computing devices and/or computing system. The notification may indicate an improvement in the detected events (e.g., a subsidence of symptoms, an efficacy of a medical treatment on the patient, a reduction in user complaints, or the like).

Control chart generation module 410 may store computer-readable instructions that, when executed, cause processing circuitry 106 to generate a time-based control chart (e.g., control chart 200) and/or a proportion-based control chart (e.g., control chart 300). In some examples, control chart generation module 410 stores computer-readable instructions that, when executed, cause processing circuitry 106 to display the time-based control chart and/or the proportion-based control chart, e.g., on UI 402.

Processing circuitry 106 may generate a control chart (e.g., control chart 200 and/or control chart 300) by generating a control chart defined by an x-axis (e.g., event date 204) and a y-axis (e.g., TBE 202, proportionality 302). Processing circuitry 106 may then place one or more detected events within the control chart. Processing circuitry 106 may place each of the one or more detected events within the area defined by the x-axis and the y-axis based on the corresponding values for the x-axis metric (e.g., event date 204) and the y-axis metric (e.g., TBE 202, proportionality 302).

In some examples, the computer-readable instructions stored in control chart generation module 410, deviation identification module 408, and/or event classification module 406 may be stored in one or more applications (e.g., Minitab, Excel or spreadsheet file etc.). Processing circuitry 106 may execute the one or more applications to perform one or more functions described above.

FIG. 5 is a conceptual diagram illustrating an example user-complaint system 500 connected to the system of FIG. 1. System 500 may include network 504, computing system 102, and computing device(s) 502A-N (collectively referred to as “computing devices 502). In some examples, computing system 102, and/or computing devices 502 may be connected to cloud computing environment 110, e.g., via network 504.

System 500 may be a part of one or more computer infrastructures. Computer infrastructures may include health information systems including, but are not limited to, electronic health records (EHR) systems, practice management software, patient portals, remote patient monitoring, healthcare management systems, or the like. Computer infrastructures may include computing devices, computing systems, networks, cloud computing environments, and the like.

One or more users may access a computer infrastructure with a computing device 502. Each of computing devices 502 may correspond to one or more users and may be configured to receive complaints from the one or more users, e.g., via UI on each of computing devices 502. One or more users may enter complaints into system 500 by entering one or more inputs into computing device 502. The one or more inputs may be tactile, visual, or auditory. The complaints may include a type of a complaint, a severity of a complaint, or the like. Computing devices 502 may transmit the received complaints to computing system 102 and/or cloud computing environment 110 via network 504. Computing devices 502 may include, but are not limited to, cellphones, smartphones, tablets, laptops, personal computers, or smartwatches.

Network 504 may facilitate connection between computing system 102, computing devices 502, and/or other components of a computer infrastructure. Network 504 may include one or more computing devices, such as one or more non-edge switches, routers, hubs, gateways, security devices such as firewalls, intrusion detection, and/or intrusion prevention devices, severs, cellular base stations and nodes, wireless access points, bridges, cable modems, application accelerators, or other network devices. Network 504 may include one or more networks administered by service providers and may thus form part of a large-scale public network infrastructure, e.g., the Internet. Network 504 may provide computing devices and systems, such as those illustrated in FIG. 5, access to the Internet and/or an intranet, and may provide a communications framework that allows computing devices 502 and computing system 102 to communicate with each other. Network 504 may isolate some of the data flowing from devices external to the private network for security purposes. In some examples, the communications between computing devices 502, computing system 102, and/or cloud computing environment 110 are encrypted.

Computing system 102 may receive the complaints entered by the one or more users via network 504. Computing system 102 may identify adverse events in the complaints, e.g., in accordance with the process discussed in greater detail in FIG. 4. For user-complaint system 500, adverse events may correspond to an increase in the frequency of complaints, a decrease in the time between complaints, an increase in severity of complaints, or the like. For example, processing circuitry 106 of computing system 102 may identify the adverse events in response to the execution of computer-readable instructions stored in memory 108.

Computing system 102 may output one or more time-based control charts 200 and/or proportion-based control charts 300 to at least one of computing devices 502. In some examples, computing system 102 may, in response to a determination of the presence of one or more adverse events, send a notification to at least one of computing devices 502, e.g., in accordance with the process discussed herein. In some examples, computing system 102 may send the notification to an administrator system 506. Administrator system 506 may control user-complaint system 500 and at least portions of one or more computer infrastructures. In some examples, computing system 102 will output, based on the determination of adverse events, one or more recommended changes to portions of system 500 and/or computer infrastructures. In some examples, administrator system 506 may automatically perform the one or more recommended changes in response to a determination of an adverse event by computing system 102.

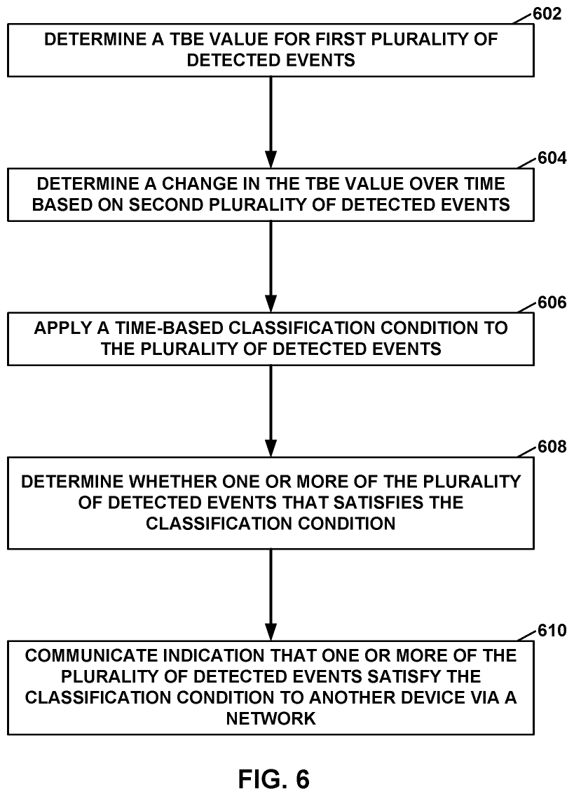

FIG. 6 is a flowchart illustrating an example process of monitoring changes in detected TBE events. While the example process illustrated in FIG. 6 is described with respect to computing system 102 of FIGS. 1 and 4, the example process may be performed by one or more other computing devices, computing systems, and/or cloud computing environments.

Computing system 102 may determine a TBE value for a first plurality of detected events (602). Computing device 112 and/or computing system 102 may detect the events and may store the detected events in memory 108 of computing system 102. In some examples, computing system 102 determines the TBE value by determining a time-between-event value (e.g., in days, hours, seconds etc.) between a first detected event and a second detected event. In some examples, computing system 102 determines an average TBE value for the first plurality of detected events. Computing system 102 may establish the determined TBE value as a BL value. Computing system 102 may determine an UCL value and an LCL value based at least in part on the determined TBE value. In some examples, computing system 102 determines the UCL value and/or the LCL value based at least in part on the determined TBE value and reference data. In some examples, computing system 102 may generate and output one or more control charts based at least in part on the determined TBE value, the UCL value, the LCL value, and the first plurality of detected events.

Computing system 102 may determine a change in the TBE value over time based on a second plurality of detected events (604). In some examples, computing system 102 may adjust the one or more control charts to illustrate the second plurality of detected events and/or changes in the BL, UCL, and/or LCL values.

Computing system 102 may apply a time-based classification condition to the plurality of detected events (606). The plurality of detected events may include the first plurality of detected events and the second plurality of detected events. The time-based rareness classification condition may include a time-based UCL, a time-based LCL, and/or a trend of the plurality of detecting events.

Computing system 102 may determine if one or more of the plurality of detected events satisfies a classification condition (608). In some examples, the classification condition may be the UCL and/or the LCL. Computing system 102 may determine that one or more of the plurality of detected events satisfies the classification condition by determining that the one or more detected events have a TBE value less than the LCL or greater than the UCL. In some examples, computing system 102 may determine that one or more detected events satisfies a classification condition by determining that a trend across the plurality of detected events is a decreasing trend. In some examples, computing system 102 may assign an “adverse event” identification to the one or more detected events based on the determination. Computing system 102 may assign an “adverse event” identification and/or an “adverse trend: identification if the TBE value is less than LCL or if the TBE values for consecutive detected events are trending towards zero.

Computing system 102 may communicate an indication that one or more of the plurality of detected events satisfy the classification condition to another device via a network (610). In some examples, computing system 102 may communicate the indication to another device within a same computer infrastructure or within different computer infrastructures. The indication may include, but are not limited to, notifications of an occurrence of an adverse event, one or more control charts, one or more recommended changes, a notification that the one or more recommended changes have been made, e.g., by computing system 102, or the like. In some examples, computing system 102 may output, as an indication, one or more proportion-based control charts with labelled adverse events (e.g., via indicator 216) identified by computing system 102 to satisfy one or more classification conditions.

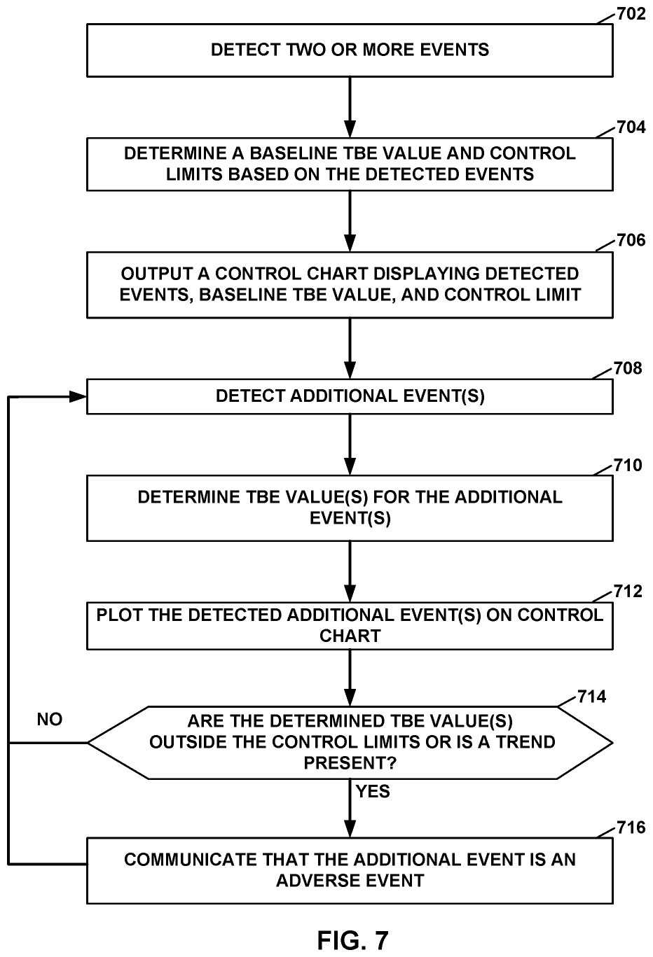

FIG. 7 is a flowchart illustrating an example process of determining the presences of changes in TBE values that satisfy a classification condition. While the example process illustrated in FIG. 7 is described with respect to computing system 102 of FIGS. 1 and 4, the example process may be performed by one or more other computing devices, computing systems, and/or cloud computing environments.

Computing system 102 detects two or more events (702). Computing system 102 may be configured to detect a monitored event such as, but is not limited to, occurrence of a symptom, delivery of a medical therapy, or a user complaint. In some examples, computing system 102 may detect the two or more events through one or more computing devices (e.g., computing device 112, computing devices 502, or the like). In some examples, computing system 102 may detect the two or more events based on input from one or more users, e.g., via an UI of computing system 102 and/or the one or more computing devices.

Computing system 102 may determine a BL TBE value and control limits based on the detected events (704). The control limits may include an UCL TBE value and a LCL TBE value. Computing system 102 may determine the control limits based at least in part on the BL TBE value and reference data. Reference data may include prior detected events of one or more users (e.g., user 114), prior detected events of other individuals, subject-matter expert opinion, or the like. In some examples, computing system 102 may determine, based on the reference data, the control limits as a percentage offset from the BL value (e.g., ±30% of the BL TBE value or a function of the BL TBE value). In some examples, such as if the monitored event is represented by low-fidelity data, computing system 102 may determine the control limits by detecting additional events.

Computing system 102 may output a control chart (e.g., control chart 200) displaying detected events, BL TBE value, and control limits (706). control chart 200 is a time-based control chart and has the event date of the detected events as an X-axis and the corresponding TBE value as a Y-axis. Computing system 102 may plot the BL value and control limits onto control chart 200 based on the corresponding TBE values. Computing system 102 may define each of the detected events by the event date and corresponding TBE value and may plot each detected event on control chart 200 by the event date and TBE value. In some examples, only a UCL or a LCL may be plotted on control chart 200, depending on the type of monitored event.

Computing system 102 may detect additional event(s) (708), determine TBE value(s) for the additional event(s) (710), and plot detected additional event(s) on control chart 200 (712). Computing system 102 may determine if one or more of the detected additional event(s) have determined TBE value(s) outside of a control limit (714). For example, computing system 102 may determine if the determined TBE value(s) show a trend (e.g., an adverse trend), are greater than the UCL TBE value or are less than the LCL TBE value. If computing system 102 determines that the determined TBE value(s) are within the control limits (“NO” branch of 714), computing system 102 may detect additional event(s) (708) and repeat the process.

If computing system 102 determines that the determined TBE value(s) are outside of the control limits or that a trend is present (“YES” branch of 714), computing system 102 may communicate (e.g., to computing device 112, cloud computing environment 110, computing devices 502, network 504, administrator system 506, or the like) that one or more of the detected additional event(s) is an adverse event (716). Computing system 102 may then detect additional event(s) (708) and repeat the process.

The techniques of this disclosure may be implemented in a wide variety of computing devices, medical devices, or any combination thereof. Any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory that is tangible. The computer-readable storage media may be referred to as non-transitory. A server, client computing device, or any other computing device may also contain a more portable removable memory type to enable easy data transfer or offline data analysis.

The techniques described in this disclosure, including those attributed to various modules and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated, discrete logic circuitry, or other processing circuitry, as well as any combinations of such components, remote servers, remote client devices, or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components. For example, any module described herein may include electrical circuitry configured to perform the features attributed to that particular module, such as fixed function processing circuitry, programmable processing circuitry, or combinations thereof.

The techniques described in this disclosure may also be embodied or encoded in an article of manufacture including a computer-readable storage medium encoded with instructions. Instructions embedded or encoded in an article of manufacture including a computer-readable storage medium encoded, may cause one or more programmable processors, or other processors, to implement one or more of the techniques described herein, such as when instructions included or encoded in the computer-readable storage medium are executed by the one or more processors. Example computer-readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or any other computer readable storage devices or tangible computer readable media. The computer-readable storage medium may also be referred to as storage devices.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

It should be noted that medical device system 100, and the techniques described herein, may not be limited to use in a human patient. In alternative examples, medical device system 100 may be implemented in non-human patients, e.g., primates, canines, equines, pigs, and felines. These other animals may undergo clinical or research therapies that my benefit from the subject matter of this disclosure. Various examples are described herein, such as the following examples.

Example 1: a computing system comprising: a memory; processing circuitry coupled to the memory, the processing circuitry configured to: determine a time-between-event (TBE) value for a first plurality of events detected by a computing device; determine a classification condition based on the determined TBE value and reference data stored in the memory, the reference data comprising information corresponding to prior detected events; determine a change in the TBE value over time for a second plurality of events detected by the computing device; based on the determination of the change in the TBE value, apply the classification condition to a plurality of detected events, wherein the plurality of detected events comprises the first plurality of events and the second plurality of events; and determine whether one or more of the plurality of detected events satisfies the classification condition; and communications circuitry configured to: communicate to another device via a network that the one or more of the plurality of detected events satisfies the classification condition.

Example 2: the computing system of example 1, wherein the processing circuitry is further configured to predict a future detected event based on the classification condition.

Example 3: the computing system of example 2, wherein the communications circuitry is further configured to communicate to the another device, via the network, the predicted future detected event.

Example 4: the computing system of any of examples 1-3, wherein the processing circuitry is further configured to output a control chart displaying the plurality of detected events, an average TBE value over time, and the classification condition.

Example 5: the computing system of example 4, wherein to output the control chart, the processing circuitry is configured to determine the average TBE value over time and the classification condition based on at least two detected events.

Example 6: the computing system of any of examples 1-5, wherein the processing circuitry is further configured to identify one or more detected events of the plurality of detected events that satisfy the classification condition but do not satisfy a proportion-based rareness classification condition.

Example 7: the computing system of any of examples 1-6, wherein the computing device comprises a medical device.

Example 8: the computing system of any of examples 1-7, wherein the classification condition comprises a lower control limit.

Example 9: the computing system of example 8, wherein to determine whether the one or more of the plurality of detected events satisfies the classification condition, the processing circuitry is further configured to determine, for each of the one or more detected events, whether a TBE value corresponding to the respective detected event is less than the lower control limit.

Example 10: the computing system of any of examples 8 and 9, wherein to determine the classification condition, the processing circuitry is further configured to: determine a baseline value based on the TBE value for the first plurality of events; and determine the lower control limit based at least partially on the baseline value.

Example 11: the computing system of example 10, wherein to determine the lower control limit based at least partially on the baseline value, the processing circuitry is further configured to determine the lower control limit based on the baseline value and the reference data.

Example 12: the computing system of any of examples 8-11, wherein the lower control limit has a value that is 70 percent of or greater than the TBE value for the first plurality of events.

Example 13: the computing system of any of examples 8-12, wherein the lower control limit has a value that is less than 70 percent of the TBE value for the first plurality of events.

Example 14: the computing system of any of examples 1-7, wherein the classification condition comprises a trend of changes in TBE values for the plurality of events over time, and wherein to determine whether the one or more of the plurality of detected events satisfies the classification condition, the processing circuitry is further configured to determine whether the trend of changes in TBE values is a decreasing trend.

Example 15: the computing system of any of examples 1-14, wherein the processing circuitry is further configured to adjust the classification condition based at least partially on the change in the TBE value.

Example 16: the computing system of any of examples 1-15, wherein the first plurality of events comprises at least two events.

Example 17: the computing system of any of examples 1-16, wherein the reference data comprises the information corresponding to prior events detected by one or more of the computing device or one or more other computing devices.

Example 18: a computing system comprising: a memory; processing circuitry coupled to the memory, the processing circuitry configured to: determine a time-between-event (TBE) value for a first plurality of symptom occurrences experienced by a patient that are detected by a medical device; determine a classification condition based on the determined TBE value and reference data stored in the memory, the reference data comprising information corresponding to prior detected symptom occurrences; determine a change in the TBE value over time for a second plurality of symptom occurrences experienced by the patient that are detected by the medical device; based on the determination of the change in the TBE value, apply the classification condition to a plurality of detected symptom occurrences, wherein the plurality of detected symptom occurrences comprises the first plurality of detected symptom occurrences and the second plurality of detected symptom occurrences; and determine whether one or more of the plurality of detected symptom occurrences satisfies the classification condition; and communications circuitry configured to: communicate to another device via a network that the one or more of the plurality of detected symptom occurrences satisfies the classification condition.

Example 19: the computing system of example 18, wherein the processing circuitry is further configured to predict a future symptom occurrence based on the application of the classification condition.

Example 20: the computing system of example 19, wherein the communication circuitry is further configured to communicate to another device, via the network, the predicted future symptom occurrence.

Example 21: the computing system of any of examples 18-20, wherein the processing circuitry is further configured to output a control chart displaying the plurality of detected symptom occurrences, an average TBE value over time, and the classification condition.

Example 22: the computing system of example 21, wherein to output the control chart, the processing circuitry is configured to determine the average TBE value over time and the classification condition based on two detected symptom occurrences and on the reference data.

Example 23: the computing system of any of examples 18-22, wherein the processing circuitry is further configured to identify one or more of the plurality of detected symptom occurrences that satisfy the classification condition but do not satisfy a proportion-based rareness classification condition.

Example 24: the computing system of any of examples 18-23, wherein the medical device comprises an implantable medical device.

Example 25: the computing system of any of examples 18-24, wherein the classification condition comprises a lower control limit.

Example 26: the computing system of example 25, wherein to determine whether the one or more of the plurality of detected symptom occurrences satisfies the classification condition, the processing circuitry is further configured to determine, for each of the one or more detected symptom occurrences, whether a TBE value corresponding to the respective detected symptom occurrence is less than the lower control limit.

Example 27: the computing system of any of examples 25 and 26, wherein to determine the classification condition, the processing circuitry is further configured to:

determine a baseline value based on the TBE value for the first plurality of symptom occurrences; and determine the lower control limit based at least partially on the baseline value.

Example 28: the computing system of example 27, wherein to determine the lower control limit based at least partially on the baseline value, the processing circuitry is further configured to determine the lower control limit based on the baseline value and the reference data.

Example 29: the computing system of any of examples 25-28, wherein the lower control limit has a value that is 70 percent of or greater than the TBE value for the first plurality of symptom occurrences.

Example 30: the computing system of any of examples 25-28, wherein the lower control limit has a value that is less than 70 percent of the TBE value for the first plurality of symptom occurrences.

Example 31: the computing system of any of examples 18-24, wherein the classification condition comprises a trend of changes in TBE values for the plurality of events over time, and wherein to determine whether the one or more of the plurality of detected events satisfies the classification condition, the processing circuitry is further configured to determine whether the trend of changes in TBE values is a decreasing trend.

Example 32: the computing system of any of examples 18-31, wherein the processing circuitry is further configured to adjust the classification condition based at least partially on the change in the TBE value.

Example 33: the computing system of any of examples 18-32, wherein the first plurality of symptom occurrences comprises at least two symptom occurrences.

Example 34: the computing system of any of examples 18-33, wherein the reference data comprises the information corresponding to prior detected symptom occurrences experienced by one or more of the patient or one or more other persons.

Example 35: a method comprising: determining, by processing circuitry of a computing system, a time-between-event (TBE) value for a first plurality of events detected by a computing device; determining, by the processing circuitry, a classification condition based on the determined TBE value and reference data stored in memory of the computing system, the reference data comprising information corresponding to prior detected events; determining, by the processing circuitry, a change in the TBE value over time for a second plurality of events detected by the computing device; applying, by the processing circuitry and based on the determined change in the TBE value, the classification condition to a plurality of detected events, wherein the plurality of detected events comprises the first plurality of events and the second plurality of events; determining, by the processing circuitry, whether one or more of the plurality of detected events satisfies the classification condition; and communicating, by communications circuitry of the computing system, an indication that the one or more of the plurality of detected events satisfies the classification condition to another device via a network.

Example 36: the method of example 35, further comprising predicting, by the processing circuitry, a future detected event based on the application of the classification condition.

Example 37: the method of any of examples 35 and 36, further comprising outputting, by the processing circuitry, a control chart displaying the plurality of detected events, an average TBE value over time, and the classification condition.

Example 38: the method of example 37, wherein outputting the control chart further comprises determining, by the processing circuitry, the average TBE value over time and the classification condition based on two or more detected events.

Example 39: the method of any of examples 37 and 38, further comprising communicating, by the communications circuitry, the control chart to another device via the network.

Example 40: the method of any of examples 35-39, wherein the computing device comprises a medical device.

Example 41: the method of any of examples 35-40, wherein the classification condition comprises a lower control limit.

Example 42: the method of example 41, wherein determining whether the one or more of the plurality of detected events satisfies the classification condition further comprises determining, by the processing circuitry and for each of the one or more detected events, whether a TBE value corresponding to the respective detected event is less than the lower control limit.

Example 43: the method of any of examples 41 and 42, wherein determining the classification condition comprises: determining, by the processing circuitry, a baseline value based on the TBE value for the first plurality of events; and determining, by the processing circuitry, the lower control limit based at least partially on the baseline value.

Example 44: the method of example 43, wherein determining the lower control limit based at least partially on the baseline limit comprises determining, by the processing circuitry, the lower control limit based on the baseline value and the reference data.

Example 45: the method of any of examples 41-44, wherein the lower control limit has a value that is 70 percent of or greater than the TBE value for the first plurality of events.

Example 46: the method of any of examples 41-44, wherein the lower control limit has a value that is less than 70 percent of the TBE value for the first plurality of events.

Example 47: the method of any of examples 35-40, wherein the classification condition comprises a trend of changes in TBE values for the plurality of events over time, and determining whether the one or more of the plurality of detected events satisfies the classification condition comprises determining, by the processing circuitry, whether the trend of changes in TBE values is a decreasing trend.

Example 48: the method of any of examples 35-47, further comprising adjusting, by the processing circuitry, the classification condition based at least partially on the change in the TBE value.

Example 49: the method of any of examples 35-48, wherein the first plurality of events comprises at least two events.

Example 50: the method of any of examples 35-49, wherein the reference data comprises the information corresponding to prior events detected by one or more of the computing device or one or more other computing devices.

Example 51: a computer readable storage medium storing instructions that, when executed by processing circuitry within a computing system, cause the processing circuitry to perform the method of any of examples 35-50.

Example 52: A computing system comprising: a memory; processing circuitry coupled to the memory, the processing circuitry configured to: determine a time-between-event (TBE) value for a plurality of events detected by a computing device; determine a classification condition based on the determined TBE value and reference data stored in the memory, the reference data comprising information corresponding to prior detected events; apply the classification condition to the plurality of detected events; and determine whether one or more of the plurality of detected events satisfies the classification condition; and communications circuitry configured to: communicate to another device via a network that the one or more of the plurality of detected events satisfies the classification condition.

Example 53: the computing system of example 52, wherein the processing circuitry is further configured to predict a future detected event based on the application of the classification condition.

Example 54: the computing system of example 53, wherein the communications circuitry is further configured to communicate to the another device, via the network, the predicted future detected event.

Example 55: the computing system of any of examples 52-54, wherein the processing circuitry is further configured to output a control chart displaying the plurality of detected events, an average TBE value over time, and the classification condition.

Example 56: the computing system of example 55, wherein to output the control chart, the processing circuitry is configured to determine the average TBE value over time and the classification condition based on two detected events.

Example 57: the computing system of any of examples 52-56, wherein the processing circuitry is further configured to identify one or more detected events of the plurality of detected events that satisfy the classification condition but do not satisfy a proportion-based rareness classification condition.

Example 58: the computing system of any of examples 52-57, wherein the computing device comprises a medical device.

Example 59: the computing system of any of examples 52-58, wherein the classification condition comprises a lower control limit.

Example 60: the computing system of example 59, wherein to determine whether the one or more the plurality of detected events satisfies the classification condition, the processing circuitry is further configured to determine, for each of the one or more detected events, whether a TBE value corresponding to the respective detected event is less than the lower control limit.

Example 61: the computing system of any of examples 59 and 60, wherein to determine the classification condition, the processing circuitry is further configured to: determine a baseline value based on the TBE value for the first plurality of events; and determine the lower control limit based at least partially on the baseline value.

Example 62: the computing system of example 61, wherein to determine the lower control limit based at least partially on the baseline value, the processing circuitry is configured to determine the lower control limit based on the baseline value and the reference data.

Example 63: the computing system of any of examples 59-62, wherein the lower control limit has a value that is 70 percent of or greater than the TBE value for the first plurality of events.

Example 64: the computing system of any of examples 59-63, wherein the lower control limit has a value that is less than 70 percent of the TBE value for the first plurality of events.

Example 65: the computing system of any of examples 52-58, wherein the classification condition comprises a trend of changes in TBE values for the plurality of events over time, and wherein to determine whether the one or more of the plurality of detected events satisfies the classification condition, the processing circuitry is further configured to determine whether the trend of changes in TBE values is a decreasing trend.

Example 66: the computing system of any of examples 52-65, wherein the processing circuitry is further configured to adjust the classification condition based at least partially on the change in the TBE value.

Example 67: the computing system of any of examples 52-66, wherein the plurality of events consists of two events.

Example 68: the computing system of any of examples 52-67, wherein the reference data comprises the information corresponding to prior events detected by one or more of the computing device or one or more other computing devices.

Example 69: A computing system comprising: a memory; processing circuitry coupled to the memory, the processing circuitry configured to: determine a classification condition based on a determined TBE value; based on the TBE value, apply the classification condition to a plurality of detected events; and determine whether one or more of a plurality of detected events satisfies the classification condition.

Example 70: The computing system of Example 69 configured with any of Examples 1-69.

Various examples have been described herein. Any combination of the described operations or functions is contemplated. These and other examples are within the scope of the following claims.

What is claimed is:

1. A computing system comprising:
a memory;
processing circuitry coupled to the memory, the processing circuitry configured to:
determine a time-between-event (TBE) value for a first plurality of events detected by a computing device;
determine a classification condition based on the determined TBE value and reference data stored in the memory, the reference data comprising information corresponding to prior detected events;
determine a change in the TBE value over time for a second plurality of events detected by the computing device;
based on the determined change in the TBE value, apply the classification condition to a plurality of detected events, wherein the plurality of detected events comprises the first plurality of events and the second plurality of events; and
determine whether one or more of the plurality of detected events satisfies the classification condition; and
communications circuitry configured to:
communicate to another device via a network that the one or more of the plurality of detected events satisfies the classification condition.

2. The computing system of claim 1, wherein the processing circuitry is further configured to predict a future detected event based on the application of the classification condition.

3. The computing system of claim 2, wherein the communications circuitry is further configured to communicate to another device, via the network, the predicted future detected event.

4. The computing system of claim 1, wherein the classification condition comprises a lower control limit.

5. The computing system of claim 4, wherein to determine whether the one or more the plurality of detected events satisfies the classification condition, the processing circuitry is further configured to determine, for each of the one or more detected events, whether a TBE value corresponding to the respective detected event is less than the lower control limit.

6. The computing system of claim 4, wherein to determine the classification condition, the processing circuitry is further configured to:
determine a baseline value based on the TBE value for the first plurality of events; and
determine the lower control limit based at least partially on the baseline value.

7. The computing system of claim 6, wherein to determine the lower control limit based at least partially on the baseline value, the processing circuitry is configured to determine the lower control limit based on the baseline value and the reference data.

8. The computing system of claim 1, wherein the classification condition comprises a trend of changes in TBE values for the plurality of events over time, and wherein to determine whether the one or more of the plurality of detected events satisfies the classification condition, the processing circuitry is further configured to determine whether the trend of changes in TBE values is a decreasing trend.

9. The computing system of claim 1, wherein the processing circuitry is further configured to adjust the classification condition based at least partially on the change in the TBE value.

10. The computing system of claim 1, wherein the first plurality of events comprises at least two events.

11. The computing system of claim 1, wherein the reference data comprises the information corresponding to prior events detected by one or more of the computing device or one or more other computing devices.

12. A computing system comprising:
a memory;
processing circuitry coupled to the memory, the processing circuitry configured to:
determine a time-between-event (TBE) value for a first plurality of symptom occurrences experienced by a patient that are detected by a medical device;
determine a classification condition based on the determined TBE value and reference data stored in the memory, the reference data comprising information corresponding to prior detected symptom occurrences;
determine a change in the TBE value over time for a second plurality of symptom occurrences experienced by the patient that are detected by the medical device;
based on the determination of the change in the TBE value, apply the classification condition to a plurality of detected symptom occurrences, wherein the plurality of detected symptom occurrences comprises the first plurality of detected symptom occurrences experienced and the second plurality of detected symptom occurrences; and
determine whether one or more of the plurality of detected symptom occurrences satisfies the classification condition; and
communications circuitry configured to:
communicate to another device via a network that the one or more of the plurality of detected symptom occurrences satisfies the classification condition.

13. The computing system of claim 12, wherein the processing circuitry is further configured to predict a future symptom occurrence based on the application of the classification condition.

14. The computing system of claim 12, wherein the communication circuitry is further configured to communicate to the another device, via the network, the predicted future symptom occurrence.

15. The computing system of claim 12, wherein the classification condition comprises a lower control limit.

16. The computing system of claim 15, wherein to determine whether the one or more of the plurality of detected symptom occurrences satisfies the classification condition, the processing circuitry is further configured to determine, for each of the one or more detected symptom occurrences, whether a TBE value corresponding to the respective detected symptom occurrence is less than the lower control limit.

17. The computing system claim 15, wherein to determine the classification condition, the processing circuitry is further configured to:
determine a baseline value based on the TBE value for the first plurality of symptom occurrences; and
determine the lower control limit based at least partially on the baseline value.

18. The computing system of claim 12, wherein the classification condition comprises a trend of changes in TBE values for the plurality of events over time, and wherein to determine whether the one or more of the plurality of detected events satisfies the classification condition, the processing circuitry is further configured to determine whether the trend of changes in TBE values is a decreasing trend.

19. The computing system of claim 12, wherein the first plurality of symptom occurrences comprises at least two symptom occurrences.

20. A method for operating processing circuitry of a computing device comprising:
determining, by the processing circuitry of the computing system, a time-between-event (TBE) value for a first plurality of events detected by a computing device;
determining, by the processing circuitry, a classification condition based on the determined TBE value and reference data stored in memory of the computing system, the reference data comprising information corresponding to prior detected events;
determining, by the processing circuitry, a change in the TBE value over time for a second plurality of events detected by the computing device;
applying, by the processing circuitry and based on the determined change in the TBE value, the classification condition to a plurality of detected events, wherein the plurality of detected events comprises the first plurality of events and the second plurality of events;
determining, by the processing circuitry, whether one or more of the plurality of detected events satisfies the classification condition; and
communicating, by communications circuitry of the computing system, an indication that the one or more of the plurality of detected events satisfies the classification condition to another device via a network.
